(19) United States
(12) Patent Application Publication
(10) Pub. No.: US 2018/0043133 A1
(43) Pub. Date: Feb. 15, 2018

(54) EXPANDABLE SHEATH AND METHODS OF USE

(71) Applicant: Advanced Cardiology Engineering Solutions, LLC, San Jose, CA (US)

(72) Inventor: James Wong, San Jose, CA (US)

(21) Appl. No.: 15/677,975

(22) Filed: Aug. 15, 2017

Related U.S. Application Data
(60) Provisional application No. 62/375,141, filed on Aug. 15, 2016.

(51) Int. Cl. A61M 25/01; A61M 25/00

(52) U.S. Cl. CPC A61M 25/0152; A61M 25/001; A61M 25/0108; A61M 25/0041; A61M 25/0045; A61M 25/005; A61M 25/0074; A61M 25/0097; A61M 2025/0024; A61M 2025/0025; A61M 25/0158; A61F 2/2436

(57) ABSTRACT

Disclosed herein are expandable introducer sheaths and methods of making and using the same. The sheaths minimize trauma to a patient's vasculature by allowing for temporary expansion of a portion of the sheath state after passage of the device. The sheath includes a rolled inner member having a detachable flap structure at its distal tip that facilitates expansion of the sheath lumen to increase diameters. Also, the flap structure reduces the number of layers at the tip, lowering push forces. An elastomeric free end of the tip helps reduce push and retrieval forces for balloons and implants. The expandable sheath includes an elastic outer layer that compresses the inner member and flap back into the rolled, compressed condition after the passage of the device.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims priority benefit of U.S. Provisional Application No. 62/375,141, filed Aug. 15, 2016, the entirety of which is hereby incorporated by reference herein.

### BACKGROUND

### Field of the Invention

The present application relates in some aspects to embodiments of a sheath for use with catheter-based technologies to deploy a prosthetic device, such as a heart valve or other implant, into the patent's vasculature.

### Description of the Related Art

Endovascular delivery catheter assemblies are used to implant prosthetic devices, such as a prosthetic heart valve, at locations inside the body that are not readily accessible by surgery or where access without surgery is desirable. For example, mitral, tricuspid, aortic, and/or pulmonary prosthetic valves can be delivered to a treatment site using minimally invasive surgical techniques, including transcatheter delivery methods.

An expandable sheath can be used to safely introduce a delivery apparatus into a patent's vasculature (e.g., the femoral artery). An expandable sheath generally has an elongated sleeve that is inserted into the vasculature and a housing that contains one or more sealing valves that allow a delivery apparatus to be placed in fluid communication with the vasculature with minimal blood loss. A conventional introducer sheath typically requires a tubular loader to be inserted through the seal in the housing to provide an unobstructed path through the housing for the prosthetic implant, such as a heart valve mounted on a balloon catheter. A conventional loader extends from the proximal end of the introducer sheath, and therefore decreases the available working length of the delivery apparatus that can be inserted through the sheath and into the body.

Conventional methods of accessing a vessel, such as the femoral artery, including dilating the vessel using multiple dilators or sheaths that progressively increase the diameter prior to introducing the delivery system. This repeated insertion and vessel dilation can increase the amount of time the procedure takes, as well as the risk of damage to the vessel.

Radially expanding intravascular sheaths reduce the overall profile of the sheath to reduce the damage to the vessel. Such introducer sheaths tend to have complex mechanisms, such as ratcheting mechanisms that maintain the shaft or sheath in an expanded configuration once a device with a larger diameter than the sheath's original diameter is introduced.

However, delivery and/or removal of the prosthetic devices and other materials to or from a patient can still poses a threat to a patient. Furthermore, accessing the vessel remains a challenge due to the relatively large profile of the delivery system; bending or kinking can cause longitudinal and radial tearing of the vessel during insertion. The delivery system can additionally dislodge calcified plaque within the vessel, posing a risk of clots caused by the dislodged plaque. The addition of radially expanding properties can also hinder a practitioner's ability to push the introducer sheath without it bending or kinking. There remains a need for further improvements in introducer sheaths for the endovascular system using implanting heart valves and other prosthetic devices.

### SUMMARY

Disclosed herein are expandable sheaths and methods of making and using an expandable flat-sheet-rolled-shaft low profile sheath.

In certain embodiments, an expandable sheath is disclosed. The expandable sheath includes an elongated inner member defining a central lumen, a first longitudinal free edge, and a second longitudinal free edge along both a first circumferential portion and a second circumferential portion, the first circumferential portion including a proximal free end, the second circumferential portion distal to the first circumferential portion, wherein the elongated inner member is configured to overlap at the first and second longitudinal free edges in a rolled configuration, wherein the second circumferential portion is positioned at least partially between the overlapping first and second free longitudinal edges. The expandable sheath also includes and an outer elastomeric member extending around the elongated inner member and configured to bias the elongated inner member. The elongated inner member further includes a distal tip, the distal tip including a flap extending from the first free longitudinal edge and at least to the second free longitudinal edge of the second circumferential portion of the elongated inner member.

In certain embodiments, the flap is configured to slide circumferentially over an outer surface of the second circumferential portion when the elongated inner member is biased by the elastic outer member. In certain embodiments, the second circumferential portion has a distal edge extending longitudinally at least to a proximal edge of the flap. In certain embodiments, the proximal edge of the flap extends over the distal edge of the second circumferential portion onto an outer surface of the second circumferential portion. In certain embodiments, the flap includes a longitudinal section of the second circumferential portion cut along the second longitudinal edge. In certain embodiments, the longitudinal section is cut circumferentially from the distal end of the second circumferential portion. In certain embodiments, the proximal edge of the flap extends circumferentially from the longitudinal section. In certain embodiments, the proximal edge of the flap extends proximally from the longitudinal section. In certain embodiments, the distal tip includes an elastomeric end extending from a distal end of the elongated inner member. In certain embodiments, the elastomeric end has a distally tapering shape. In certain embodiments, the expandable sheath includes a marker embedded in the distal tip of the elongated inner member. In certain embodiments, the elongated inner member includes a slit extending proximally from a distal edge of the second circumferential portion. In certain embodiments, the elongated inner member and the distal tip are integrally formed.

In certain embodiments, a method of an expandable sheath is disclosed. The method includes forming a rolled configuration in an elongated inner member by forming an overlap along a first longitudinal edge and a second longitudinal edge of the elongated inner member so that a first circumferential portion is positioned at least partially between the longitudinal edges in the rolled configuration, forming a flap on a distal tip of the inner member so that the flap extends from a first longitudinal edge of the inner member at least to a second longitudinal edge of the inner member, and covering the elongated inner member with an elastomeric outer member.

In certain embodiments, forming the flap includes extending the flap circumferentially over the outer surface of the first circumferential portion. In certain embodiments, forming the flap includes forming a proximal edge on the flap that extends over a distal edge and onto an outer surface of the second circumferential portion. In certain embodiments, the flap is formed at least partially by cutting a longitudinal section from the second circumferential portion. In certain embodiments, the flap is formed at least partially by attaching an overlap extension to the longitudinal section. In certain embodiments, the method includes attaching an elastomeric end to a distal end of the elongated inner member. In certain embodiments, the method further includes forming a tapered shape into the elastomeric end.

In certain embodiments, a method of delivering a prosthetic device is disclosed. The method incudes positioning an expandable sheath within a vascular system of a patient, introducing a prosthetic device into a lumen of the expandable sheath, advancing the prosthetic device through the lumen of the expandable sheath such that the prosthetic device exerts a radially outward force on an inner surface of an inner member of the expandable sheath and locally partially unrolls the inner member into an expanded configuration, advancing the prosthetic device further through the lumen to a distal tip of the expandable sheath and causing a free end of the distal tip to slide circumferentially over an outer surface of a first circumferential portion of the expandable sheath to locally enlarge the lumen in response to radial pressure exerted by passage of the prosthetic device, and at least partially collapsing the inner member at the distal tip after the prosthetic device has passed out of the lumen of the expandable sheath.

In certain embodiments, the method includes advancing the prosthetic device therethrough. In certain embodiments, at least partially collapsing the inner member includes sliding the free end of the flap of the distal tip circumferentially over the outer surface of the first circumferential portion to locally reduce the lumen.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings reference numbers are used to indicate specific design element of the inventions.

FIG. 1 is a perspective view of an open polymer sheet according to an illustrative embodiment.

FIG. 2 is a perspective view of an inner shaft according to an illustrative embodiment.

FIG. 3 is a front view of distal end of an inner shaft according to an illustrative embodiment.

FIG. 4 is a front view of an inner shaft according to an illustrative embodiment.

FIG. 5 is a side view of the sheath according to an illustrative embodiment.

FIG. 6 is cross section view of a distal end of a sheath according to an illustrative embodiment.

FIG. 7A is a top view of a distal end of an open polymer sheet according to an illustrative embodiment.

FIG. 7B is a top view of a distal end of an open polymer sheet according to an illustrative embodiment.

FIG. 7C is a top view of a distal end of a first open polymer sheet and a second open polymer sheet according to an illustrative embodiment.

FIG. 8 is a top view of a distal end of a first open polymer sheet and a second open polymer sheet according to an illustrative embodiment.

FIG. 9 is a top view of a first open polymer sheet and a second open polymer sheet according to an illustrative embodiment.

FIG. 10 is a front view of an inner shaft and outer jacket according to an illustrative embodiment.

FIG. 11 is a partial cross-sectional view of an inner shaft and outer jacket according to an illustrative embodiment.

FIG. 12 is a perspective view of a section of an inner shaft and outer jacket according to an illustrative embodiment.

FIG. 13 is a side view of a sheath showing a portion of an outer jacket removed according to an illustrative embodiment.

FIG. 14 is a partial-cross sectional view of a sheath showing a portion of an outer jacket removed according to an illustrative embodiment.

FIG. 15 is a perspective view of a section of an inner shaft according to an illustrative embodiment.

FIG. 16 is a perspective view of a section of an inner shaft according to an illustrative embodiment.

FIG. 17 is a side view of a sheath and a hub according to an illustrative embodiment.

FIG. 18 is a perspective view of a section of an inner shaft according to an illustrative embodiment.

FIG. 19 is a side view of a section of a sheath coupled to a hub according to an illustrative embodiment.

FIG. 20 is a side view of a sheath coupled to a hub according to an illustrative embodiment.

FIG. 21 is a side view of an introducer according to an illustrative embodiment

FIG. 22 is a cross-sectional view showing a section of an introducer positioned in a section of a sheath according to an illustrative embodiment

FIG. 23 is a perspective view of an introducer coupled according to an illustrative embodiment.

FIG. 24 is a perspective view of a sheath coupled to a hub according to an illustrative embodiment.

FIG. 25 is a perspective view of an introducer positioned within a sheath coupled to a hub according to an illustrative embodiment.

FIG. 26 is a top view of a flat sheet according to an illustrative embodiment.

FIG. 27 is a top view of a flat sheet according to an illustrative embodiment.

FIG. 28 is a perspective view of a section of an inner shaft according to an illustrative embodiment.

FIG. 29 is a perspective view of a section of an inner shaft according to an illustrative embodiment.

FIG. 30 is a perspective view of a section of an inner shaft and suture according to an illustrative embodiment.

FIG. 31 is a perspective view of an inner shaft and suture according to an illustrative embodiment.

FIG. 32 is a perspective view of a coil according to an illustrative embodiment.

### DETAILED DESCRIPTION

In some embodiments, expandable sheaths as disclosed herein can be used to deliver a prosthesis device through a patient's vasculature to a procedure/implantation site within the body. The sheath can be constructed to be highly expandable and collapsible in the circumferential/radial direction, while also minimizing the wall thickness of the sheath to minimize puncture size and/or accommodate a larger profile of the delivery system. In some embodiments, an expandable sheath can include any one, two, or more features as described in the description herein.

In some embodiments, an expandable sheath can be made of a rolled shaft from a sheet, such as a rectangular sheet in some cases. The sheet can be flat or substantially flat, and/or include macro or micro surface features, e.g., ridges, wells, and/or microstructures in some embodiments. In some embodiments, an expandable flat-sheet-rolled-shaft low profile sheath has an expandable flat-sheet-rolled-shaft design that can be made using a flat polymer sheet that can have smooth inner and outer-facing surfaces. The expandable flat-sheet-rolled-shaft design can be made by coating the flat sheet inner surface with hydrophilic coating, rolling and forming a shaft with edges of the sheet overlapping along its long axis. The length of the flat sheet can form a length of the shaft. The width of the flat sheet can form a circumference of the shaft with additional portions of the width forming overlapping edges.

The low profile expandable flat-sheet-rolled-shaft can include an elastic polymer outer jacket covering its entire length. The elastic polymer outer jacket can be sealed at its distal and/or proximal end to provide a uniformly concentric sealing feature along the entire length of the flat-sheet-rolled-shaft. Elastic properties of the jacket can allow it to instantly or almost instantly recover the shaft to its low-profile configuration after an implant delivery system is advanced through a lumen of the shaft. The elastic polymer wall thickness can be engineered to protect against internal systolic blood pressure forces being exerted on the inner wall of the sheath.

The overlapping free edges of the shaft allow the shaft to be expanded radially with no constraints of structural radial force or friction along the length of the shaft of the expandable flat-sheet-rolled-shaft to facilitate the placement of large bulky implants, collapsible heart valves and other medical devices through lumen of the shaft. In some embodiments, the rolled shaft does not include any folded or creased sections that reversibly expand asymmetrically along only part of a circumference of the tube.

In some embodiments, the expandable flat-sheet-rolled-shaft low profile sheath design has an expandable distal tip and a hub with hemostasis valve at a proximal end. In some embodiments, the sheath has a flared and/or tapered proximal end.

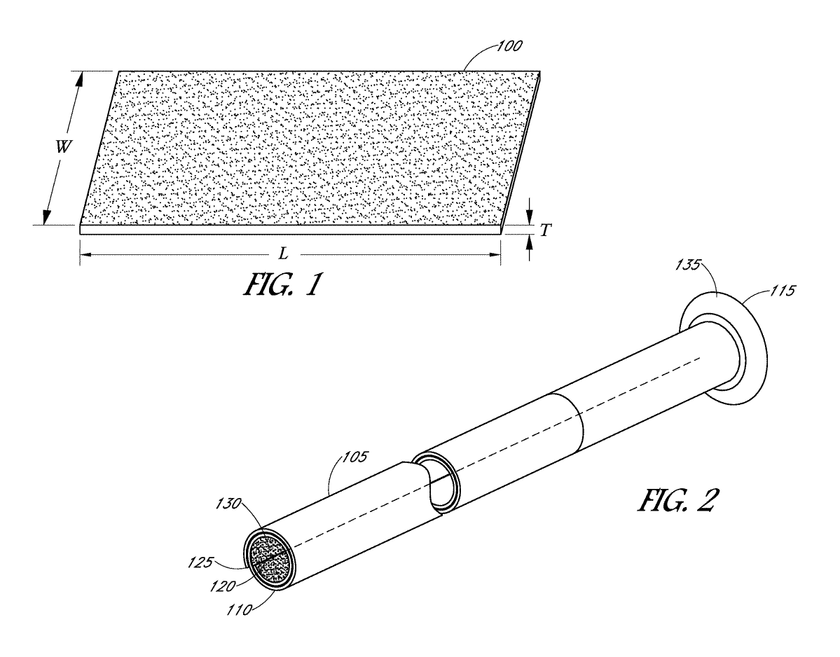

FIG. 1 depicts an embodiment of a flat polymer sheet 100 having a width W, a length L and a wall thickness T. In some embodiments, the wall thickness T can be between about 0.009 inches and about 0.012 inches. In some embodiments, the flat polymer sheet 100 can be rolled along its width W to form a rolled shaft. In some embodiments, the length L of the flat polymer sheet 100 can become a length of a rolled-shaft. In some embodiments, the width W can define a circumference of the rolled-shaft plus and overlapping edges widths of the rolled-shaft. In some embodiments, a surface of the flat sheet 100 can be coated with a hydrophilic coating. In some embodiments, the flat sheet and hydrophilic coating can be cured.

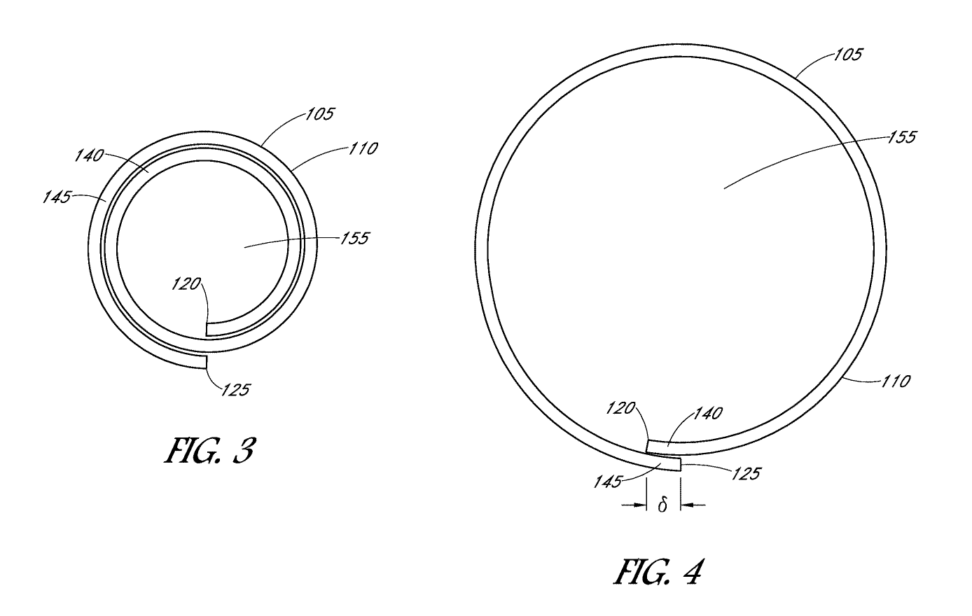

FIG. 2 depicts an embodiment of a rolled shaft 105 formed from the flat polymer sheet 100. The rolled shaft 105 can have a distal end 110 and a proximal end 115. The rolled shaft 105 can include an interior free edge 120 and an exterior free edge 125 extending at least partially along the length of the shaft. In some embodiments, the proximal end 115 can include a flared region 135. In some embodiments, the proximal and distally facing edges do not meet or contact each other head-on. The flared region 135 can be thermoformed. The interior edge 120 and exterior edge 125 can be free lateral ends that overlap to form the rolled shaft 105 In some embodiments, the interior edge 120 and exterior edge 125 can be free to move relative to one another.

In some embodiments, the rolled shaft 105 can be formed by rolling the flat sheet 100 with a support rod to form a rolled sheet. After the flat sheet 100 is rolled, heat can be applied to form the rolled shaft 105. In some embodiments, the flat sheet can be rolled to form a rolled shaft such that the hydrophilic coating is on an interior and/or exterior section of the rolled shaft.

In some embodiments, the sheet 100 can be over a PTFE coated or stainless steel mandrel to form the rolled shaft 105.

FIG. 3 depicts the distal end of the rolled shaft 105. As shown, the rolled shaft 105 includes overlapping sections forming the inner layer 140 and outer layer 145. In FIG. 3, the interior edge 120 is generally aligned with the outer edge 125. As shown in FIG. 3, the polymer sheet 100 has undergone two revolutions along its width W to form the rolled shaft 105. In some embodiments, the configuration of FIG. 3 is the “normal” or “resting” configuration of the rolled shaft 105 when no foreign bodies (e.g., an implant) are introduced into the rolled shaft 105 and the rolled shaft is at its minimum diameter. In some embodiments, the polymer sheet 100 is rolled about or at least about 1.2, 1.35, 1.5, 1.75, 2, 2.25, 2.5, 2.75, 3, 3.25, 3.5, or more revolutions in its normal or resting configuration. The rolled shaft 105 can be expanded or partially unrolled upon the introduction of one or more foreign bodies into a lumen 155 of the rolled shaft 105. The inner lumen 155 can be defined by the radially inward-facing sidewall of the inner layer 145.

The lumen 105 can be, in some embodiments, less than 6 French, 6 French, 8 French, 10 French, 12 French, 14 French, 16 French, 18 French, or more than 18 French in diameter at one or more positions along the rolled shaft 105.

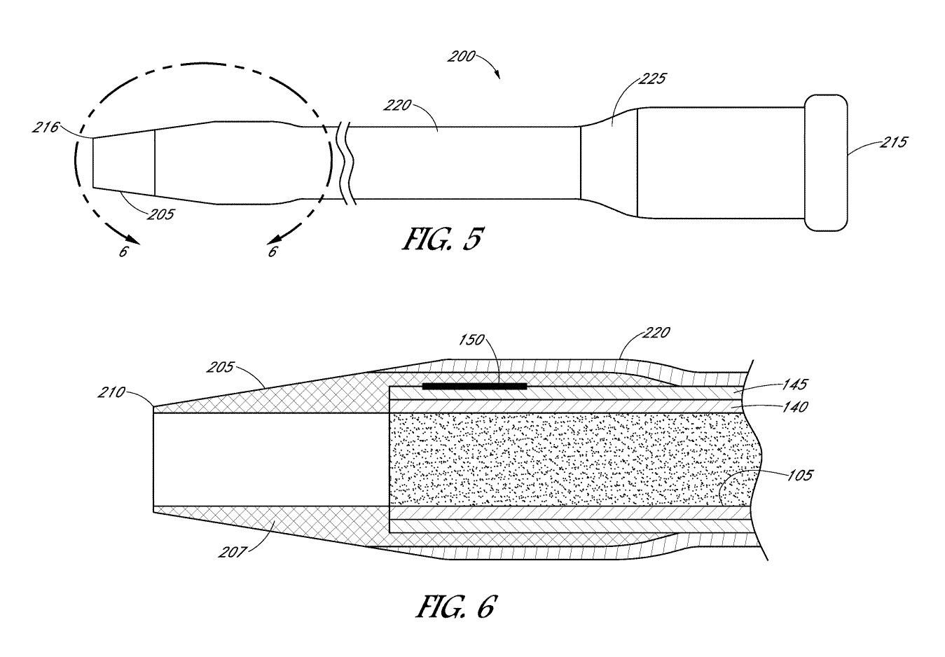

FIG. 4 shows an expanded or partially unrolled view of the rolled shaft 105. As shown in FIG. 4, the inner lumen 155 can have a greater diameter than in the normal or resting configuration. The rolled shaft 105 can be reversibly radially expanded due to the introduction of one or more foreign bodies introduced into the inner lumen 155. FIG. 4 depicts a maximum amount of expansion or unrolling of the rolled shaft 105, where the shaft is rolled to slightly more than one revolution, such as less than about 1.10, 1.09, 1.08, 1.07, 1.06, 1.05, 1.04, 1.03, 1.02, or 1.01 revolutions, but still more than exactly one revolution such that the shaft does not completely and irreversibly unroll, and is prevented from doing so by the dimensions of the system, outer sheath, and maximum permitted size of the implant that is allowed to be placed within the shaft. In some embodiments, after formation of the rolled shaft 105, the interior edge 120 unrolls beyond the exterior edge 125 to open the rolled shaft 105. In some embodiments, the rolled shaft 105 will have a minimum length 6 over which the exterior edge 125 extends beyond the interior edge 120 to form an overlap. In some embodiments, the length 6 can be the length of the inner layer 140 when the rolled shaft has undergone the maximum amount of expansion or unrolling. In some embodiments, the inner shaft 105 can recover to the normal configuration after expansion if the foreign body is removed from the rolled shaft 105. In some embodiments, the rolled shaft 105 can expand and retract repeatedly.

FIG. 5 depicts an embodiment of a sheath assembly 200. The sheath assembly 200 includes a distal end 210 and a proximal end 215. The sheath assembly 200 includes a distal tip 205 at the distal end 210. An interior of the sheath assembly 200 is formed from the rolled shaft 105 (not shown in FIG. 5). In some embodiments, the sheath assembly 200 includes an outer jacket 220. The outer jacket 220 can at least partially encase the rolled shaft 105. In some embodiments, the outer jacket 220 can encase an entire length of the rolled shaft 105. As shown in FIG. 5, the sheath assembly 200 can include a flared region 225 corresponding to the flared region 135 of the rolled shaft 100 of FIG. 3.

An inner lumen of the tip 205 can be less than 6 French, 6 French, 8 French, 10 French, 12 French, 14 French, 16 French, 18 French, or more than 18 French in diameter at one or more positions along the rolled shaft 105.

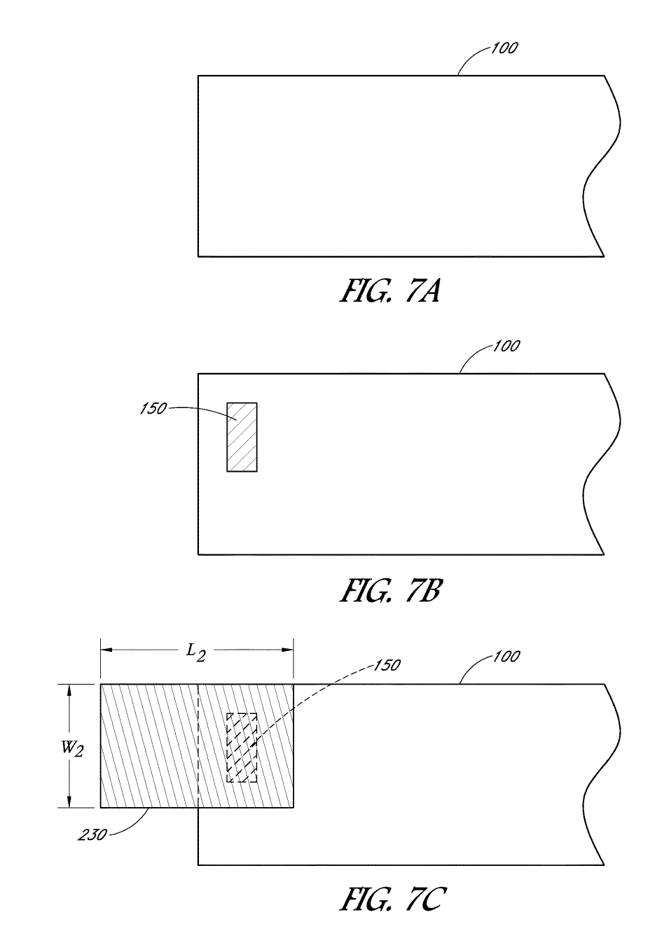

FIG. 6 depicts a cross-sectional view of a distal section of the sheath assembly 200 taken along line 6-6 of FIG. 5. As shown in FIG. 6, the rolled shaft 105 can include an inner layer 140 and an outer layer 145. In some embodiments, a marker band 150 can be positioned on an exterior surface of the outer layer 145. A portion of the distal tip 205 can be positioned between the outer layer 145 of the rolled shaft 105 and the outer jacket 220. The portion of the distal tip extending beyond the rolled shaft 105 can form a tab or flap 207. In some embodiments, the marker band 150 is positioned between the outer layer 145 and the outer jacket 220. In some embodiments, the marker band 150 is a radiopaque marker band.

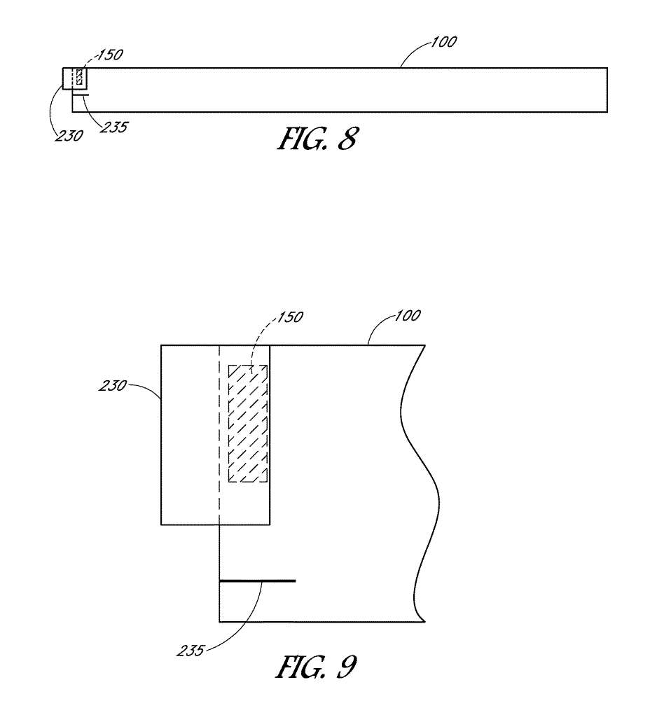

FIGS. 7A-7C depict an example of a process for preparing a rolled shaft 105 and distal tip 205.

FIG. 7A depicts a distal section of a flat polymer sheet 100. As depicted in FIG. 7B, the marker band 150 can be affixed to a distal portion of the flat sheet 100. In some embodiments, the marker band 150 is attached to the flat sheet 100 using a soldering iron. As shown in FIG. 7C, a second flat sheet 230 (e.g., a distal tab or flap) can be placed so that at least a portion of the second flat sheet 230 extends over the first flat sheet 100 and marker band 150. The second flat sheet 230 can have a width W2 that is less than the width W of the first flat sheet 100 and a length L2 that is less than the length L of the first flat sheet 100. A distal end of the second flat sheet 230 can extend distally beyond the distal end of the first flat sheet 100. The flat sheet 230 can have a smaller thickness than the thickness T of the flat sheet 100. The flat sheet 230 can be fused with an adhesive or otherwise attached to the flat sheet 100, securing the marker band 150 in between the flat sheet 230 and flat sheet 100. The section of the flat sheet 230 extending beyond the distal end of the flat sheet 100 is not fused. After the flat sheet 230 is fused to the flat sheet 100, the flat sheet 230 and flat sheet 100 can be rolled to form the rolled shaft 105 and distal tip 205. After rolling, the width W2 of the flat sheet 230 defines a circumference of the distal tip 205. In some embodiments, the edges of the distal tip 205 do not overlap one another. In some embodiments, the edges of the distal tip 205 touch one another. In some embodiments, the distal tip 205 reinforces the column strength of the rolled shaft 105. In some embodiments, the distal tip 205 can reinforce the column strength of the rolled shaft 105 for ease of insertion and extraction.

In some embodiments, the flat sheet 100 and flat sheet 230 are rolled separately. In some embodiments, the flat sheet 100 and flat sheet 230 are rolled simultaneously. In some embodiments, one or both of the flat sheet 100 and flat sheet 230 are rolled using a PTFE coated, stainless steel, and/or Teflon mandrel. In some embodiments, the flat sheet 100 and flat sheet 230 are rolled before fusion of the flat sheet 100 to the flat sheet 230. In some embodiments, the flat sheet 100 and flat sheet 230 can be assembled and fused over the end of the flat sheet 100 while both the flat sheet 100 and flat sheet 230 are positioned on the mandrel.

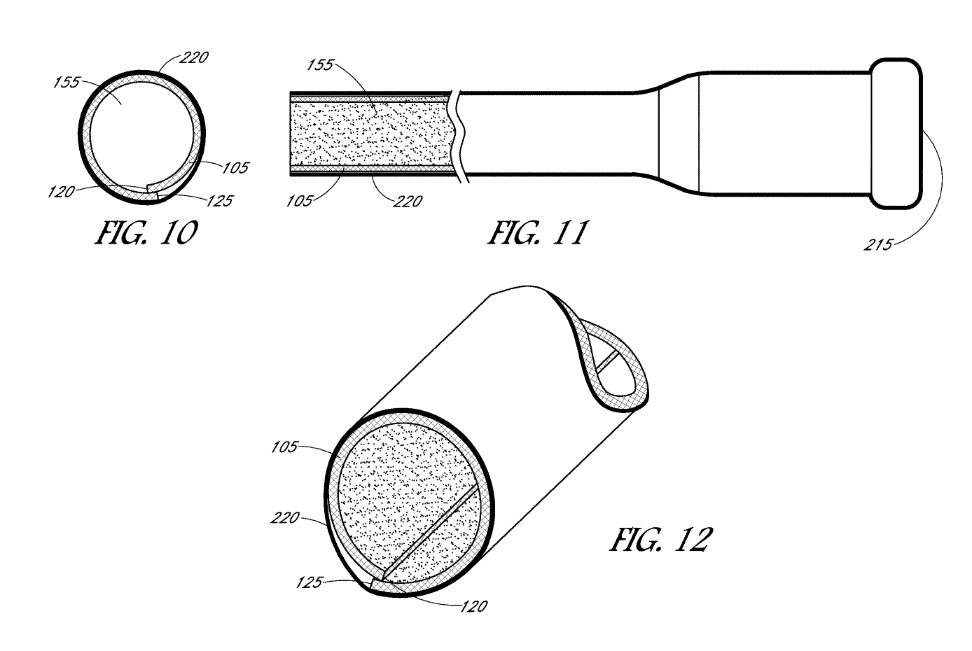

As shown in FIG. 8, in some embodiments, a slit 235 can be formed in the flat sheet 100 prior to rolling of the flat sheet 100. In some embodiments, a mark is made on the flat sheet corresponding to a desired location of the slit 235. After the mark is made, the slit 235 can be formed on the marked area. In some embodiments, the slit extends between about 1.0 mm to about 9.0 mm in the proximal direction from the distal end of the flat sheet 100. In some embodiments, after formation of the rolled shaft 105, the rolled shaft 105 can be heated at the slit 235 to temperature bond the rolled shaft 105 together at the slit 235. In some embodiments, bonding of the rolled shaft 105 together at the slit 235 can create a perforation at the slit 235. Such a perforation can rupture when a device is passed through the rolled shaft 105, which can allow for the passage of a device with less exertion of force. In some embodiments, the sheet 100 is heated to temperature bond the sheet 100 together at the slit 235 before formation of the rolled shaft 105. In some embodiments, a complementary slit is formed in the outer jacket 220 adjacent to the slit 235. In some embodiments, the slit 235 is formed after formation of the rolled shaft 105. In some embodiments, the slit 235 and a slit in the outer jacket 220 are formed simultaneously.

FIG. 9 depicts a view of a section of the polymer sheet 100, the marker band 150, and the flat sheet 230 showing the slit 235.

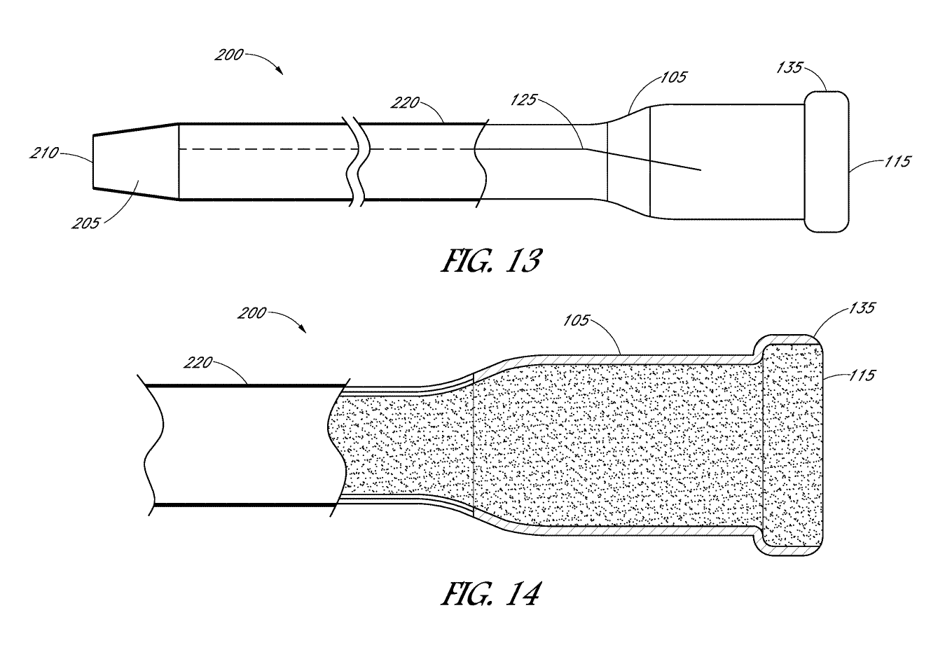

FIG. 10 depicts a cross-sectional view of the rolled shaft 105 with the outer jacket 220 in place. As shown in FIG. 7, the rolled shaft 105 is in a radially expanded configuration. The outer jacket 220 can be elastic. The outer jacket 220 can allow for limited expansion or unrolling of the rolled shaft 105. The outer jacket 220 can be configured to prevent expansion or unrolling of the rolled shaft 105 beyond a certain amount of expansion or unrolling, such as for example, the maximum amount of expansion or unrolling shown in FIG. 6.

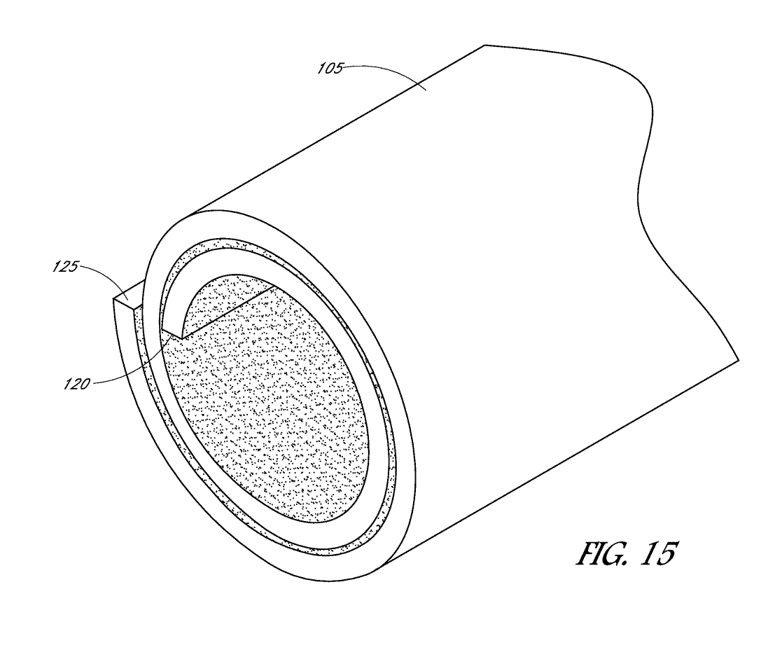

FIG. 11 depicts a partial cross-sectional view showing the rolled shaft 105 and outer jacket 220. FIG. 12 depicts a perspective view of a section of the rolled shaft 105 and outer jacket 220 in a radially expanded configuration with free interior edge 120 and free exterior edge 125 slightly overlapping more than one revolution.

FIG. 13 depicts the sheath assembly 200 showing the outer jacket 220 on only a portion of the sheath assembly 200. The exterior edge 125 is shown in dashed lines in the portion of FIG. 13 that depicts the outer jacket 220. As shown in FIG. 13, the exterior edge 125 can terminate distal to the proximal end 115. In some embodiments, the exterior edge 125 is fused to the inner layer 140 (not shown) at a proximal section of the outer jacket 220.

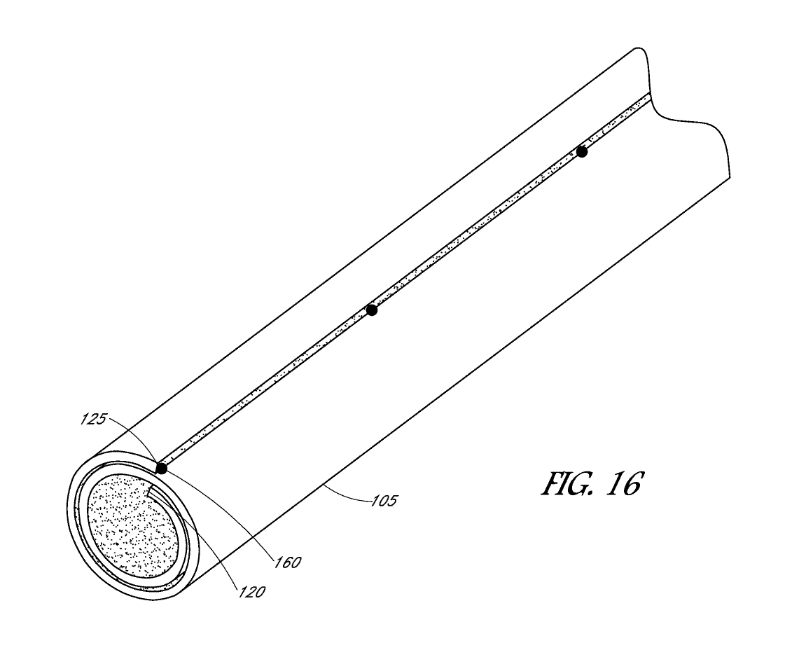

FIG. 14 depicts a portion the sheath assembly 200 showing the outer jacket 220 on only a portion of the sheath assembly 200 and a cross-sectional view of proximal section of the sheath assembly 200.

In some embodiments, a distal end of the jacket 220 extends over a portion of the distal end of the tip 205. In some embodiments, a distal end of the jacket 220 is flush with the distal end 115 of rolled shaft 105. In some embodiments, a proximal end of the jacket 220 is flush with a proximal end 115 of the rolled shaft 105.

After the outer jacket 220 is fused with the rolled shaft 105 to form the sheath assembly, the proximal end of the sheath 200 can be flared to form the flared region 225. The flared region 225 can be tapered. In some embodiments, the flared region 225 can be configured to engage a hub. For example, the flared region 225 can be received within an interior section of the hub. In some embodiments, the flared section 225 can provide clearance entry to delivery systems extending through the hub. In some embodiments, the flared region 225 can reinforce column strength of the proximal end of the sheath 200.

In some embodiments, the outer jacket 220 can provide a sealing function between the distal end 110 of the rolled shaft 105 and the proximal end 115 of the rolled shaft 105. In some embodiments, the outer jacket is coated in a hydrophilic coating.

FIG. 15 depicts a perspective view of a section of the rolled shaft 105 in a resting, radially unexpanded configuration with the flat sheet rolled on itself about two revolutions, and showing the distal end including free interior edge 120 and free exterior edge 125.

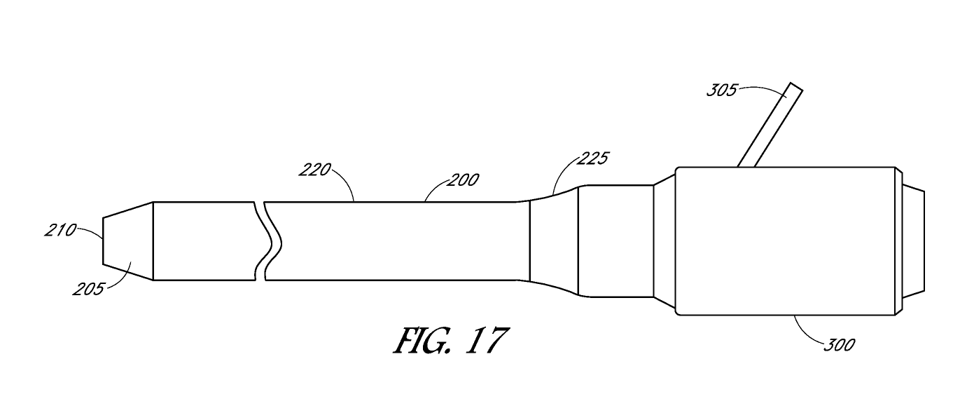

FIG. 16 depicts a perspective view of a section of the rolled shaft 105. FIG. 16 shows tacking sections 160 at discrete spaced-apart locations between the edge 125 and a section of the inner layer 140 overlapped by the edge 125. The tack can be a biocompatible water soluble adhesive, which can enhance torque performance of the shaft 105 during insertion. An introducer introduced into the shaft 105 can cause the tacked sections 160 to break apart allowing for expansion of the shaft 105.

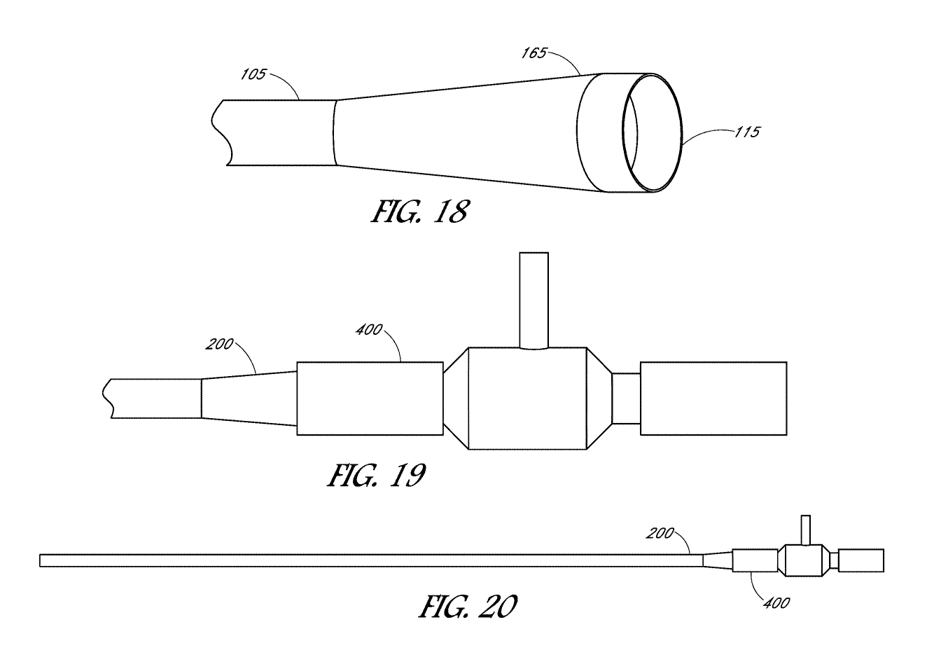

FIG. 17 depicts the sheath assembly 200 engaged with an embodiment of a hub 300. The hub 300 includes a flushing tube 305 such as an input port angled off the longitudinal axis of the sheath assembly 200. The hub 300 can include one or more hemostasis-type valves. The hub 300 can comprise a single catheter insertion port or it can comprise a plurality of catheter insertion ports. Each catheter insertion port can comprise one or more hemostasis valves, stopcocks, or the like to prevent blood leakage from the catheter. The hub 300 can further comprise one or more purge ports, which operably connect to the internal lumen of the hub and are terminated by stopcocks or other valves. In some embodiments, the hub 300 can include hub seal coupling configured to receive and secure a proximal end of the flared region 225. In some embodiments, the hub seal coupling can form a seal with the proximal end of the flared region 225.

In some embodiments, the proximal end 115 of the rolled shaft 105 can be flared prior to application of the outer jacket 220 to the rolled shaft 105. FIG. 18 depicts a perspective view of the proximal end 115 of the rolled shaft 105 with a flared section 165. The overlapping edges of the rolled shaft 105 can also be fused near the proximal end 115. After the outer jacket 220 is applied to the rolled shaft 105, both the rolled shaft 105 and outer jacket 220 can be flared to form the flared region 225.

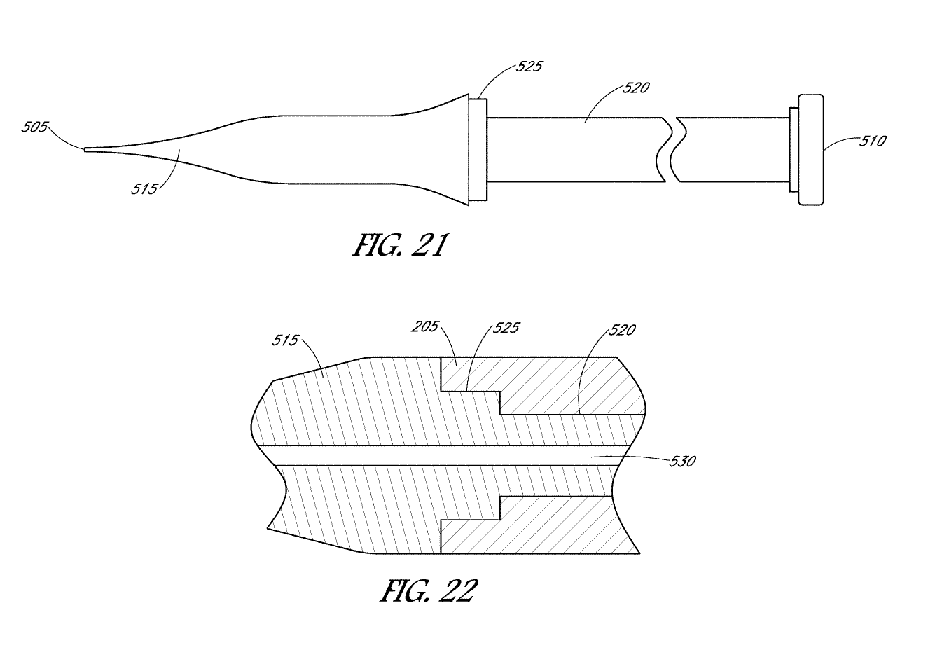

FIG. 19 depicts a proximal section of the sheath assembly 200 engaged with an embodiment of a proximal hub 400.

FIG. 20 depicts the sheath assembly 200 engaged with the hub 400. The hub 400 can include one or more hemostasis-type valves. The hub 400 can comprise a single catheter insertion port or it can comprise a plurality of catheter insertion ports. Each catheter insertion port can comprises one or more hemostasis valves, stopcocks, or the like to prevent blood leakage from the catheter. The hub 400 can further comprise one or more purge ports, which operably connect to the internal lumen of the hub and are terminated by stopcocks or other valves. In some embodiments, the hub 400 can include hub seal coupling configured to receive and secure a proximal end of the flared region 225 (not shown). In some embodiments, the hub seal coupling can form a seal with the proximal end of the flared region 225 (not shown).

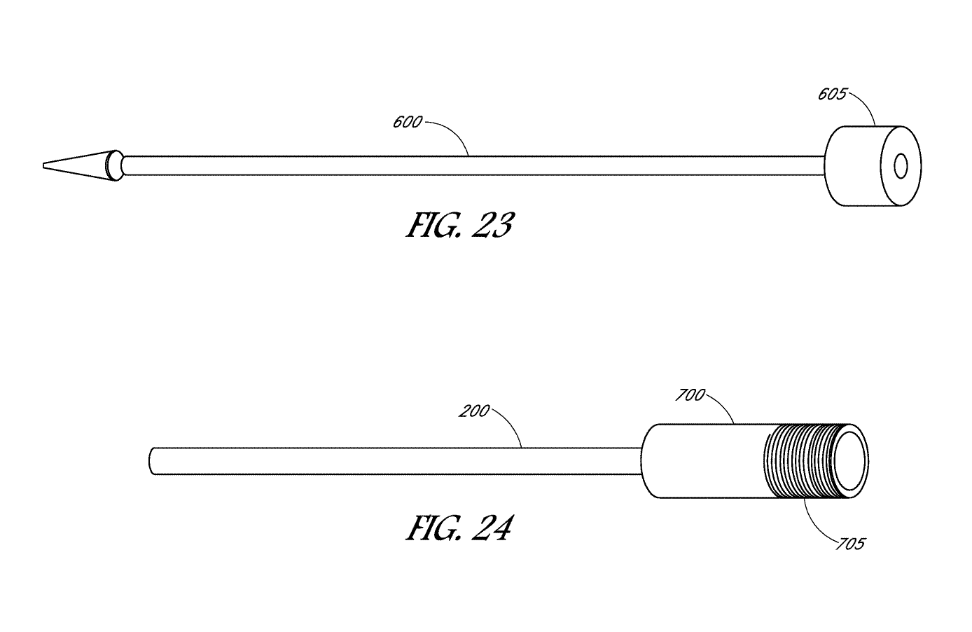

FIG. 21 depicts an embodiment of an introducer 500. The introducer 500 includes a distal end 505 and a proximal end 510. The introducer can include a distal tip 515. The introducer can also include an inner shaft 520 and an outer shaft 525. In some embodiments a proximal end of the distal tip 515 can have an outer diameter that matches an inner diameter of the distal tip 205. In some embodiments, at least a portion of the distal tip 515 can have an outer diameter between 10 French to 12 French. In some embodiments, the shaft 520 can have an outer diameter between 10 French to 12 French. In use, the distal end 505 of the introducer 500 can be introduced into the proximal end of the sheath 200 (not shown) and can be moved along the length of the sheath 200.

FIG. 22 depicts a cross-section showing a portion of the introducer 500 positioned within a portion of the sheath 200. As shown in FIG. 22, the introducer can include an inner lumen 530. The inner lumen 530 can be configured to receive a guidewire therethrough.

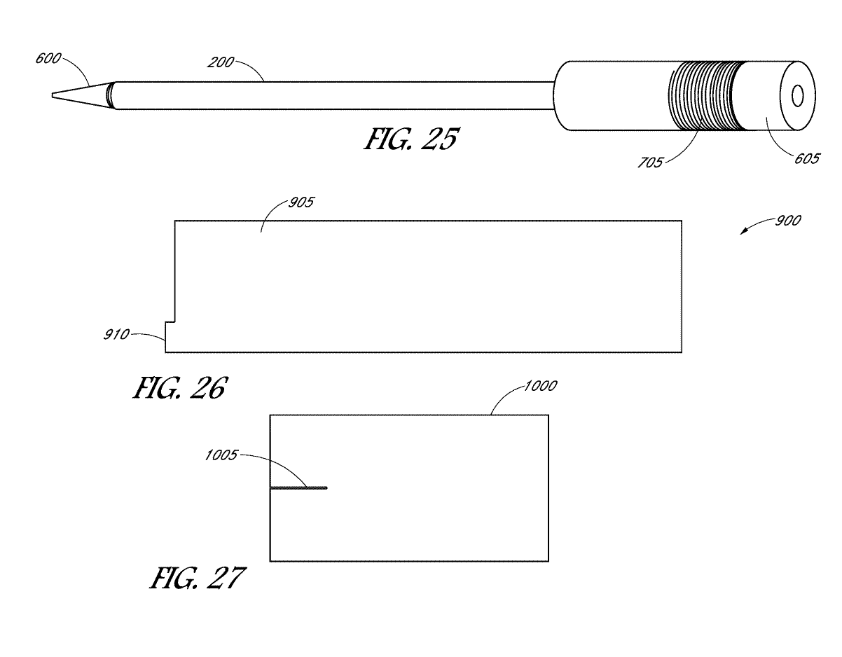

FIG. 23 depicts an embodiment of an introducer 600 having a male coupling 605 at a proximal end.

FIG. 24 depicts an embodiment of a sheath 200 and hub assembly 700. The hub assembly includes a female coupling 705 at the proximal end that can be threaded as shown.

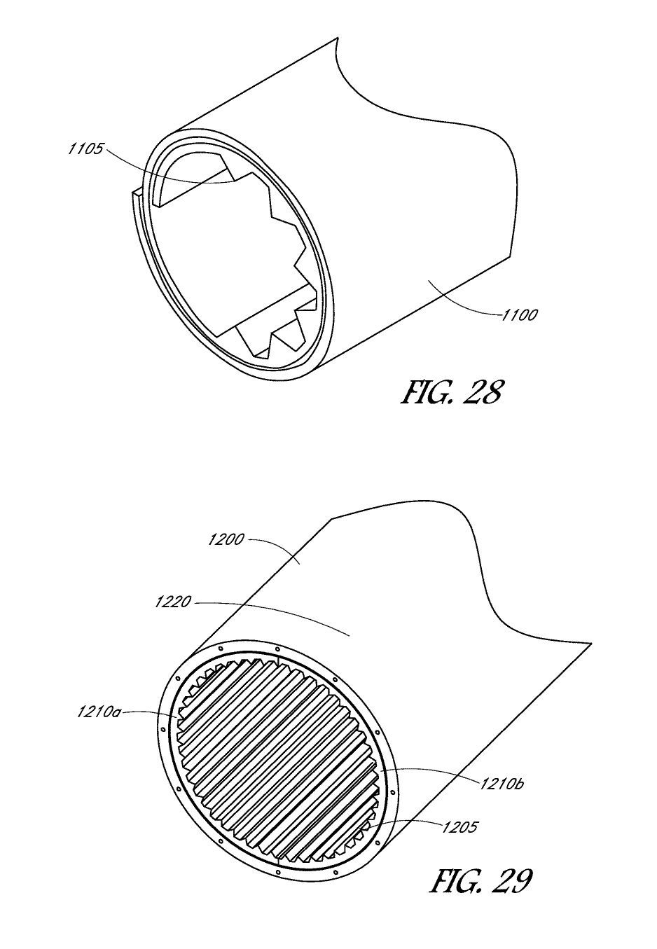

FIG. 25 depicts the introducer 600 positioned within the sheath 200 and hub assembly 700. The male coupling 605 and female coupling 705 can form an adjustable coupling. The adjustable coupling can be adjusted for flush alignment of a distal tip of the introducer with a distal end of the sheath 200.

FIG. 26 depicts an embodiment of a flat polymer sheet 900. The sheet 900 can include similar features and functions with respect to the sheet 100 as previously described. The flat polymer sheet 900 includes a first section 905 and a second section 910 integrally formed with the first section 905 extending from a distal end of the first section 905. The second section 910 can be used to form a distal tip. The flat sheet 900 can allow for formation of a rolled shaft and tip using a single flat sheet 900.

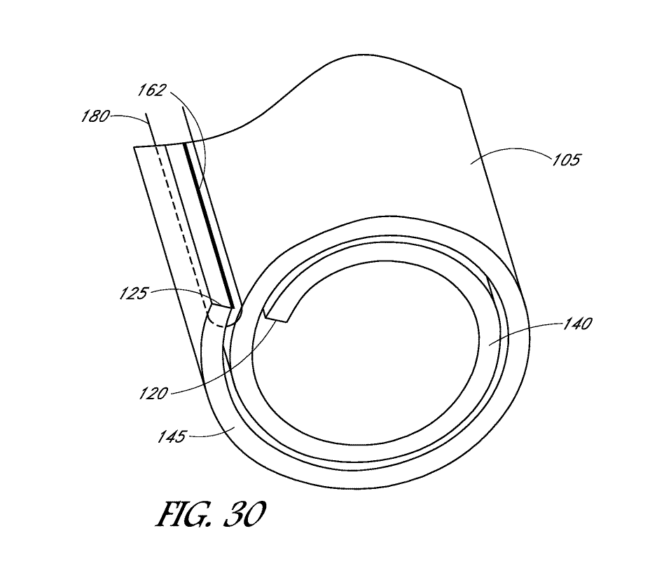

FIG. 27 depicts an embodiment of a flat sheet 1000 having a slit 1005 positioned therein. The sheet 1000 can include similar features and functions with respect to the sheet 100. In some embodiments, the slit 1005 facilitates exit and withdrawal of foreign bodies at a distal end of a rolled shaft formed using the sheet 1000. In some embodiments, the slit 1005 can reduce the force required to pass foreign bodies through the distal end of a rolled shaft formed using the sheet 1000.

FIG. 28 depicts an embodiment of a rolled shaft 1100. The rolled shaft 1100 can include similar features and functions with respect to the rolled shaft 105. The rolled shaft 1100 can further include extruded ridges 1105 along a portion of an inner surface of the rolled shaft 1100. Ridges 1105 can reduce a contact surface between overlapping layers of the shaft 1100. Ridges 1105 can reduce friction between overlapping layers of the shaft 1100. The ridges 1105 can be formed in a variety of patterns and angles to allow for motion between overlapping edges of the shaft 1100.

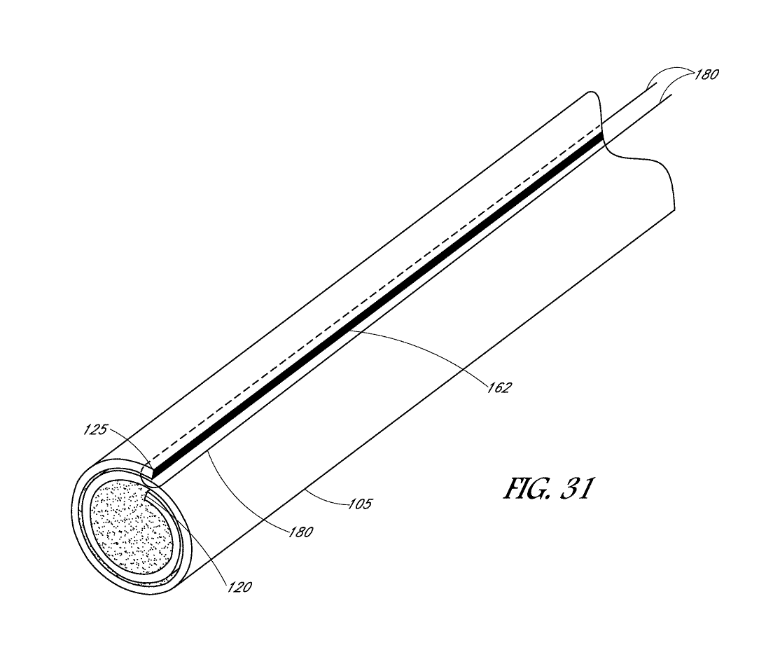

FIG. 29 depicts an embodiment of a sheath 1200. The sheath 1200 includes an outer jacket 1220 and a shaft formed of a first shaft section 1210a and a second shaft section 1210b. The shaft formed of the first shaft section 1210a and second shaft section 1210b can include similar features and functions with respect to the rolled shaft 105. The shaft section 1210a and 12010b can further include extruded ridges 1205 along an entirety of an inner surface of the first shaft section 1210a and second shaft section 1210b. The ridges 1205 can reduce friction between shaft formed of first shaft section 1210a and second shaft section 1210b and foreign bodies within the shaft. In certain embodiments, the shaft section 1210a and 1210b can be formed as an extruded tube and cut into section 1210a and 1210b. In certain embodiments, the shaft sections 1210a and 1210b can be formed as two separate sections of the tube that can then be movably coupled together and positioned within the outer jacket 1220. In certain embodiments, the shaft sections 1210a and 1210b can each be halves of a tube. In certain embodiments, the shaft sections 1210a and 1210b can separate from one another to allow space for foreign bodies to pass through the sheath 1200. In some embodiments, when the foreign bodies are removed from the sheath 1200, the sheath 1200 can return to a normal or resting configuration as shown in FIG. 29, for example, due to elasticity of the outer jacket 1120. In some embodiments, the outer jacket 1220 can include reinforced rods embedded within the outer jacket 1220. In some embodiments, the reinforced rods can be formed of Nitinol, HDPE, or any other suitable material.

FIG. 30 depicts the shaft 105 having tacking 162 or other attachment elements continuously extending along the length of the shaft 105 that attach a radially outward end of the sheet to a surface between the radially outward and radially inward end of the sheet. FIG. 30 further depicts a tether, e.g., suture 180 between the interior layer 140 and the exterior layer 145. Sections of the suture 180 between the interior layer 140 and the exterior layer 145 are shown in dashed lines. The suture 180 can extend into the proximal end of the shaft 105 and can be looped around a distal most tacking section between the interior layer 140 and exterior layer 145 and back out of the proximal end of the shaft 105. The suture 180 can be pulled to break the tacking sections.

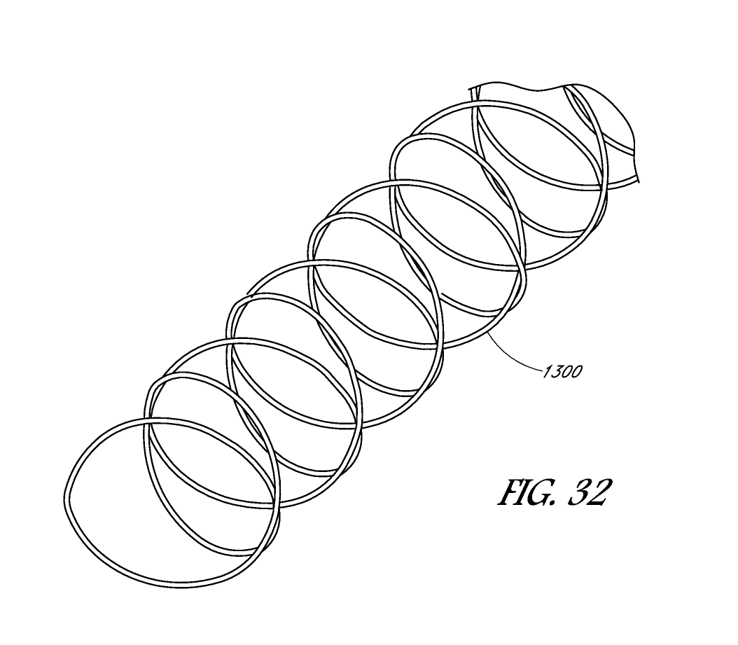

FIG. 31 depicts the shaft 105 having the tether, e.g., suture 180 between the interior layer 140 and the exterior layer 145.

FIG. 32 depicts an embodiment of one, two, or more coils 1300. In some embodiments, the coils 1300 can be positioned in between layers of the rolled shaft 105. The can be formed of, for example, a shape memory material such as nitinol and/or flat braid materials. In some embodiments, the shape and/or configuration of the coils 1300 can be temperature sensitive so that the coils 1300 are biased towards the normal or resting position of the rolled shaft 105 at body temperature.

In some embodiments, the rolled shaft 105 can include reinforced memory braids in between layers of the shaft 105. In some embodiments, the braids are formed of Nitinol.

In some embodiments, the rolled shaft 105 can include spiral ribbons positioned between the interior layer 140 and exterior layer 145 extending between the distal end 110 and the proximal end 115. In some embodiments the spiral ribbons extend continuously from the distal end 110 to the proximal end 115. In some embodiments the spiral ribbons are positioned intermittently along the length of the rolled shaft 110. In some embodiments, the spiral ribbons can include two spirals. In some embodiments, the spiral ribbons can include a left-handed spiral and a right-handed spiral. In some embodiments spirals of the spiral ribbons can overlap at one or more points along the spiral ribbon. In some embodiments, the spiral ribbons can include intermittent spaces between each point at which the spirals overlap. In some embodiments, the spirals may be oriented at one or more predetermined angles at each point at which the spirals overlap. In some embodiments, the spiral ribbons can provide increased torsional strength to the wall of the rolled shaft 105. In some embodiments, the spiral ribbons can provide improved steering to the rolled shaft 105.

In some embodiments, the spiral ribbons can facilitate greater exertion of torque to the rolled shaft 105.

In some embodiments, the distal tip 205 can expand to have a larger diameter or cross-sectional area than the maximum diameter or cross-sectional area of the lumen 155. In some embodiments, the distal tip 205 can be formed of a sheet that is thinner than the sheet 100 forming the rolled shaft 105, which can allow for the formation of a larger cross-section. In some embodiments, the distal tip 205 does not include any overlapping sections such that when the rolled shaft 105 expands, the distal tip 205 expands to create a larger cross-sectional area than the maximum diameter of the lumen 155. In some embodiments, the material of the distal tip 205 can be elastic or expandable. In some embodiments, the material of the distal tip 205 can be configured to be more elastic or expandable than the material of the rolled shaft 105. In some embodiments, the distal tip 205 can be configured to return to its normal or resting configuration following removal of a device from within the distal tip 205. In some embodiments, the elasticity of the distal tip 205 can reduce radial force on a device exiting the distal tip 205. In some embodiments, the elasticity of the distal tip 205 can reduce radial force on a device introduced into or withdrawn into the distal tip 205.

In some embodiments, one or more of the flat sheet 100, the flat sheet 230 (e.g., distal tab or flap) and the outer jacket 220 can include, but is not limited to, one or more of the following materials: a thermoplastic elastomer, e.g., Hytrel, Nylon, Pebax, polyether ether ketone (PEEK), composite, reinforced construction, polyester, polyurethane, polyethylene, Neusoft, or the like. In some embodiments, one or more of the flat sheet 100, the flat sheet 230 and the outer jacket 220 can include one or more radiopaque materials or can have one or more radiopaque materials attached thereto. Radiopaque materials can improve visualization under fluoroscopy. Radiopaque (RO) markers, such as marker band 150, can be affixed to the distal end of the sheath 200 to denote its distal end, the extents of the expandable region or regions, or even the orientation of the sheath 200 by mounting the RO markers asymmetrically on the tubing. The radiopaque markers comprise of bands or windings of metal such as, but not limited to, tantalum, platinum, platinum iridium, gold, and the like.

In some embodiments, the hub 300 or hub 400 can include, but is not limited to, one or more of the following materials: polycarbonate, acrylonitrile butadiene styrene (ABS), polyurethane, polyvinyl chloride, and the like. The dilator can comprise Hytrel, Pebax, polyether ether ketone (PEEK), composite, reinforced construction, polyester, polyurethane, polyethylene, or the like.

In some embodiments, the rolled shaft 105 can be formed as an extruded tube. While the extruded tube is exiting an extruder die, a cut or slit can be made along a length, e.g., the entire length of the extruded tube to form a split shaft. The extruded tube can then be cut to a desired shaft length. In some embodiments, the split shaft can be temporarily opened to a flat sheet configuration for application of a hydrophilic coating. The split shaft can then return to its rolled shaft configuration. In some embodiments, formation of the rolled shaft as an extruded tube can reduce manufacturing costs and enhance quality.

The description of certain examples of the concepts should not be used to limit the scope of the claims. Other examples, features, aspects, embodiments, and advantages will become apparent to those skilled in the art from the above description. As will be realized, the device and/or methods are capable of other different and obvious aspects, all without departing from the spirit of the inventive concepts. Accordingly, the drawings and description should be regarded as illustrative in nature and not restrictive and nonobvious features ans.

For the purpose of this description, certain aspects, advantages, and novel features of the embodiments of this disclosure are described herein. The described methods, systems, and apparatus should not be construed as limiting in any way. Instead, the present disclosure is directed toward all novel and nonobvious features and aspects of the various disclosed embodiments, alone and are not limited to any aspects in various combinations and sub-combination with one another. The disclosed methods, systems, and apparatus are not limited to any aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect, embodiment or example described herein unless incompatible therewith. All of the features disclosed in this specification (included any accompanying claims, abstract, and drawing), and/or all the steps of any method or process so disclosed, may be combined in any combination, except combinations where at least some of such features and/or steps are mutually exclusive. The invention is not restrictive to the details of any foregoing embodiments. The invention extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosed material, in whole or in part, that is said to be incorporated by reference herein is incorporated herein only to the extent that the incorporated material does not conflict with existing definitions, statements, or other disclosed material set forth in this disclosure. As such, and to the extent necessary, the disclosure as explicitly set forth supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclose material.

As used in the specification and appended claims, the singular forms, “a”, “an”, and “the” include plural referents unless the context clearly dictates otherwise. Ranges may be expressed herein as from “about” one particular value, and/or to “about” another particular value. When such a range is expressed, another aspect includes values from the one particular value and/or to the other particular value. Similarly, when values are expressed as approximations, by used of the antecedent “about”, it will be understood that the particular value forms another aspect. It will be further understood that the endpoints of each of the ranges are significant both in relation to the other endpoint, and independently of other endpoint.

“Optional” or “optionally” means that the subsequently described event or circumstances may or may not occur, and that the description includes instances where said event or circumstance occur and instance where it does not.

Throughout the description and claims of this specification, the word “comprise” and variation of the word, such as “comprising”, and “comprises”, means “including but not limited to”, and is not intended to exclude, for example, the other additives, components, integers or steps. “Exemplary” means “an example of and is not intended to convey an indication of a preferred or ideal aspect. “Such as” is not used in restrictive sense, but for explanatory purposes.

The terms “proximal” and “distal” as used herein refer to the region of the sheath, catheter, or delivery assembly. “Proximal” means that region closet to the handle of the device, while “distal” means that the region farthest away from the handle of the device.

The term “tube” or “tubular” as used herein is not meant to limit shapes to circular cross-section. Instead, tube or tubular can refer to any elongate structure with a closed cross-section and lumen extending axially there through. A tube may also have some selectively locate slits or opening therein—although it still provides enough of a closed structure to contain other component within its lumen(s).

Certain aspects, advantages and novel features of the invention are described herein in the document. It must be understood that not necessarily all such advantages may be achieved in accordance with any particular embodiment of the invention. Therefore, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the above description taken in conjunction with the accompanying drawings.

Although the foregoing embodiments of the present disclosure have been described in some detail by way of illustration and example for purposes of clarity and understanding, it will be apparent to those skilled in the art that certain changes and modification may be practiced within the spirit and scope of the present disclosure. It is intended that the scope of the present invention herein disclose should not be limited by the particular disclosed embodiments described above, but should be determined only by a fair reading of the claims that follow. Any methods disclosed herein need not be performed in the order recited. The methods disclosed herein include certain actions taken by a practitioner; however, they can also include any third-party instruction of those actions, either expressly or by implication. For example, actions such as “accessing a femoral artery” includes “instructing the accessing of a femoral artery.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. An expandable sheath, comprising:
an elongated inner member defining a central lumen, a first longitudinal free edge, and a second longitudinal free edge along both a first circumferential portion and a second circumferential portion, the first circumferential portion including a proximal free end, the second circumferential portion distal to the first circumferential portion,
wherein the elongated inner member is configured to overlap at the first and second longitudinal free edges in a rolled configuration, wherein the second circumferential portion is positioned at least partially between the overlapping first and second free longitudinal edges; and
an outer elastomeric member extending around the elongated inner member and configured to bias the elongated inner member;
wherein the elongated inner member further includes a distal tip, the distal tip including a flap extending from the first free longitudinal edge and at least to the second free longitudinal edge of the second circumferential portion of the elongated inner member.

2. The expandable sheath of claim 1, wherein the flap is configured to slide circumferentially over an outer surface of the second circumferential portion when the elongated inner member is biased by the elastic outer member.

3. The expandable sheath of claim 2, wherein the second circumferential portion has a distal edge extending longitudinally at least to a proximal edge of the flap.

4. The expendable sheath claim 3, wherein the proximal edge of the flap extends over the distal edge of the second circumferential portion onto an outer surface of the second circumferential portion.

5. The expandable sheath of claim 4, wherein the flap includes a longitudinal section of the second circumferential portion cut along the second longitudinal edge.

6. The expandable sheath of claim 5, wherein the longitudinal section is cut circumferentially from the distal end of the second circumferential portion.

7. The expendable sheath of claim 5, wherein the proximal edge of the flap extends circumferentially from the longitudinal section.

8. The expandable sheath of claim 7, wherein the proximal edge of the flap extends proximally from the longitudinal section.

9. The expandable sheath of claim 8, wherein the distal tip further comprises an elastomeric end extending from a distal end of the elongated inner member.

10. The expandable sheath of claim 9, wherein the elastomeric end has a distally tapering shape.

11. The expendable sheath of claim 10, further comprising a marker embedded in the distal tip of the elongated inner member.

12. The expandable sheath of claim 1, wherein the elongated inner member comprises a slit extending proximally from a distal edge of the second circumferential portion.

13. The expandable sheath of claim 1, wherein the elongated inner member and the distal tip are integrally formed.

14. A method of making an expandable sheath, the method comprising:
forming a rolled configuration in an elongated inner member by forming an overlap along a first longitudinal edge and a second longitudinal edge of the elongated inner member so that a first circumferential portion is positioned at least partially between the longitudinal edges in the rolled configuration;
forming a flap on a distal tip of the inner member so that the flap extends from a first longitudinal edge of the inner member at least to a second longitudinal edge of the inner member; and
covering the elongated inner member with an elastomeric outer member.

15. The method of claim 14, wherein forming the flap includes extending the flap circumferentially over the outer surface of the first circumferential portion.

16. The method of claim 15, wherein forming the flap includes forming a proximal edge on the flap that extends over a distal edge and onto an outer surface of the second circumferential portion.

17. The method of claim 16, wherein the flap is formed at least partially by cutting a longitudinal section from the second circumferential portion.

18. The method of claim 17, wherein the flap is formed at least partially by attaching an overlap extension to the longitudinal section.

19. The method of claim 18, further comprising attaching an elastomeric end to a distal end of the elongated inner member.

20. The method of claim 19, further comprising forming a tapered shape into the elastomeric end.

21. A method of delivering a prosthetic device, the method comprising:
positioning an expandable sheath within a vascular system of a patient;
introducing a prosthetic device into a lumen of the expandable sheath;
advancing the prosthetic device through the lumen of the expandable sheath such that the prosthetic device exerts a radially outward force on an inner surface of an inner member of the expandable sheath and locally partially unrolls the inner member into an expanded configuration;
advancing the prosthetic device further through the lumen to a distal tip of the expandable sheath and causing a free end of the distal tip to slide circumferentially over an outer surface of a first circumferential portion of the expandable sheath to locally enlarge the lumen in response to radial pressure exerted by passage of the prosthetic device; and
at least partially collapsing the inner member at the distal tip after the prosthetic device has passed out of the lumen of the expandable sheath.

22. The method of claim 21, further comprising advancing the prosthetic device therethrough.

23. The method of claim 21, wherein at least partially collapsing the inner member includes sliding the free end of the flap of the distal tip circumferentially over the outer surface of the first circumferential portion to locally reduce the lumen.
